(19) United States
(12) Patent Application Publication
(10) Pub. No.: US 2020/0143945 A1
(43) Pub. Date: May 7, 2020

(54) METHOD AND APPARATUS FOR ASSISTING HEALTH CASE OF USER

(71) Applicant: Korea Electronics Technology Institute, Seongnam-si (KR)

(72) Inventors: Boeun KIM, Seoul (KR); Hyedong JUNG, Seoul (KR); Saim SHIN, Seoul (KR); Jinyea JANG, Suwon-si (KR)

(73) Assignee: Korea Electronics Technology Institute, Seongnam-si (KR)

(21) Appl. No.: 16/658,567

(22) Filed: Oct. 21, 2019

(30) Foreign Application Priority Data
Nov. 6, 2018 (KR) 10-2018-0135439

(51) Int. Cl. G16H 50/20; G16H 10/60; G16H 30/20; G16H 40/63

(52) U.S. Cl. CPC G16H 50/20; G16H 40/63; G16H 30/20; G16H 10/60

(57) ABSTRACT

The present disclosure provides a method and apparatus for enabling an early treatment of a health abnormality by analyzing a health condition of a user periodically and accumulating history information of user condition data. In the health care apparatus, instructions stored in a memory causes a processor to: load history information for a user from a memory; acquire an image and a voice of the user through the audio unit; perform a diagnosis process by analyzing the history information and the image and voice information of the user, and provide reference information to the user or perform a corresponding action depending on a diagnosis result; and update the history information stored in the memory by adding the image and voice information of the user, the diagnosis result, and the reference information or information of the corresponding action.

### CROSS-REFERENCE TO RELATED APPLICATION

The present application claims priority to Korean Patent Application No. 10-2018-0135439, filed on Nov. 6, 2018, in the Korean Intellectual Property Office, which is incorporated herein in its entirety by reference.

### TECHNICAL FIELD

The present disclosure relates to a method and apparatus for managing health care of a user and, more particularly, to an apparatus and method for facilitating customized health care of the user by providing various health care information to the user.

### BACKGROUND

As the improvement of the economic level, people's view for diseases is changing and interest in aging and appearance management is increasing, and as a result, the expenditure of related costs is rapidly increasing. However, most of these costs are not covered by medical insurance schemes and are treated as potential social expenses. Thus, overall medical expenses and related hidden social expense are increasing steadily.

Meanwhile, as the population composition is getting older, the population of the aged people is increasing. In Korea, for example, the number of people aged 50 or over and the number of people aged 65 or over are expected to reach 30% and 13.2%, respectively, in 20 years.

As the proportion of the elderly in the total population increases, geriatric skin diseases are expected to increase rapidly. Although the skin diseases do not affect life, they are frequent and have a significant impact on people's economic and social activities. Accordingly, in recent years, people's interests in skin care and diseases are rapidly increasing regardless of gender and age. Therefore, there is a need for a scheme or apparatus for assisting periodic and integrated management of the skin diseases for preventing and treating them. On the other hand, because the elderly lack mobility, there is a need for an apparatus or system that facilitates the diagnosis of the elderly for another diseases other than the skin diseases without a long distance travel.

### PRIOR ART REFERENCE

### Patent Literature

Korean Laying-open Patent Publication No. 10-2016-0007754 published on 21 Jan. 2016 and entitled Method for Analyzing Skin Surface and Apparatus therefor.

### SUMMARY

An object of the present disclosure is to provide a method and apparatus for enabling an early treatment of a health abnormality by analyzing a health condition of a user periodically in everyday life, accumulating history information of user condition data, and preemptively identifying and alerting the abnormalities to the user.

Another object of the present disclosure is to provide a method and apparatus for accurately diagnosing the health abnormality using multimodal data of an image and a voice acquired at the time of a diagnosis.

Yet another object of the present disclosure is to provide a method and apparatus for allowing the user to easily manage the everyday health care by performing the diagnosis with reference to various information such as user preference, biorhythm, weather, and statistical information of other users.

According to an aspect of an exemplary embodiment, provided is a method of managing health of a user in a health care apparatus. The method includes: recognizing the user and determining whether to start a diagnosis process for the user according to a prescribed condition by the health care apparatus; loading history information for the user from a storage unit of the health care apparatus after the diagnosis process begins; acquiring an image and a voice of the user by the health care apparatus; performing the diagnosis process, by the health care apparatus, by analyzing the history information and the image and voice information of the user, and providing reference information to the user or performing a corresponding action depending on a diagnosis result; and updating the history information stored in the storage unit by adding the image and voice information of the user, the diagnosis result, and the reference information or information of the corresponding action.

The operation of determining whether to start the diagnosis process for the user may includes: photographing the image of the user and collecting the voice of the user, by the health care apparatus, to analyze a change in the image or the voice with reference to a previous image or a previous voice, respectively, existing in the history information; determining, by the health care apparatus, whether the change in the image or the voice is small compared to the previous image or the previous voice; and performing the diagnosis process, by the health care apparatus, when the change is determined to be larger than a predetermined threshold and updating the history information by adding the image and voice information of the user, by the health care apparatus, when the change is determined to be smaller than the predetermined threshold.

The operation of providing the reference information to the user or performing the corresponding action depending on the diagnosis result may includes: acquiring the image and voice of the user, and conversational information for a conversation between the user and the health care apparatus by the health care apparatus; loading underlying medical condition information of the user and the history information for the user from the storage unit of the health care apparatus; determining whether there is any abnormality, by the health care apparatus, by comparing the image, the voice, and the conversational information with a previous image existing in the history information, a previous existing in the history information, and the underlying medical condition information, respectively; and estimating, by the health care apparatus, a period required for regeneration to a normal condition and informing the estimated regeneration period to the user when no abnormality is found in the diagnosis process and making an appointment, by the health care apparatus, with a clinic when an abnormality is detected during the diagnosis process.

The operation of providing the reference information to the user or performing the corresponding action depending on the diagnosis result may includes: acquiring the image and the voice of the user, and conversational information for a conversation between the user and the health care apparatus by the health care apparatus; loading underlying medical condition information of the user, the history information for the user, personal information of the user, and external information from the storage unit of the health care apparatus; determining a current condition of the user, by the health care apparatus, by comparing the image and the voice of the user with a previous image and a previous voice existing in the history information, respectively, and comparing the conversational information with the underlying medical condition information, the personal information, and the external information; and recommending, by the health care apparatus, one or more of health care information selected from a group of information on diet, skincare, makeup, clothing, exercise, and posture suitable for the user and determined based on the personal information and the external information according to a current condition of the user.

According to an aspect of another exemplary embodiment, provided is a health care apparatus including: a memory for storing history information for a user and instructions; an audio unit acquiring a voice of the user; a camera capturing an image of the user; and a processor executing the instructions stored in the memory. When executed by the processor, the instructions causes the processor to: determine whether to start a diagnosis process for the user according to a prescribed condition when the user is recognized; load the history information for the user from the memory after the diagnosis process begins; acquire the image and the voice of the user through the audio unit and the audio unit, respectively; perform the diagnosis process by analyzing the history information and the image and voice information of the user, and provide reference information to the user or perform a corresponding action depending on a diagnosis result; and update the history information stored in the memory by adding the image and voice information of the user, the diagnosis result, and the reference information or information of the corresponding action.

The instructions may cause the processor to: acquire the image of the user through the camera and acquire the voice of the user and conversational information for a conversation between the user and the health care apparatus through the audio unit to analyze a change in the image or the voice with reference to a previous image or a previous voice, respectively, existing in the history information; determine whether the change in the image or the voice is small compared to the previous image or the previous voice; and perform the diagnosis process when the change is determined to be larger than a predetermined threshold and update the history information by adding the image and voice information of the user when the change is determined to be smaller than the predetermined threshold.

The instructions may cause the processor to: acquire the image of the user through the camera and acquire the voice of the user and conversational information for a conversation between the user and the health care apparatus through the audio unit; load underlying medical condition information of the user and the history information for the user from the memory; determine whether there is any abnormality by comparing the image, the voice, and the conversational information with a previous image existing in the history information, a previous existing in the history information, and the underlying medical condition information, respectively; and estimate a period required for regeneration to a normal condition and informing the estimated regeneration period to the user when no abnormality is found in the diagnosis process and make an appointment with a clinic when an abnormality is detected during the diagnosis process.

The instructions may cause the processor to: acquire the image of the user through the camera, the voice, and conversational information for a conversation between the user and the health care apparatus through the audio unit; load underlying medical condition information of the user, the history information for the user, personal information of the user, and external information from the memory; determine a current condition of the user by comparing the image and the voice of the user with a previous image a previous voice existing in the history information, respectively, and comparing the conversational information with the underlying medical condition information, the personal information, and the external information; and recommend one or more of health care information selected from a group of information on diet, skincare, makeup, clothing, exercise, and posture suitable for the user and determined based on the personal information and the external information according to a current condition of the user.

The present disclosure can improve an accuracy of a diagnosis by analyzing the image and voice of the user and the conversation with the user and combining the analysis results.

In particular, since symptoms appearing on a human body alone are often insufficient for accurately identifying the condition of the human body, the present disclosure improves the accuracy of the diagnosis by reflecting the analysis of the conversation with the user similarly to a medical examination by interview in a hospital.

Also, the present disclosure enables to diagnose a body rhythm and health condition of the user by detecting the tone of the voice and the change thereof which may be acquired from voice information.

The present disclosure enables to preemptively identify and cope with abnormal symptoms by automatically analyzing the routine or everyday life of the user and building up a history of such information.

Through the diagnosis using the recently acquired information and the history information, the present disclosure can perform a customized diagnosis of the abnormal conditions for the user, enable the user to perform health care related activities routinely, and provide the recommendations for enhancing health conditions of the user.

### BRIEF DESCRIPTION OF THE DRAWINGS

In order that the disclosure may be well understood, there will now be described various forms thereof, given by way of example, reference being made to the accompanying drawings, in which:

FIG. 1 is a block diagram of a health care apparatus according to an exemplary embodiment of the present disclosure;

FIG. 2 is a flowchart illustrating a user health management method according to an exemplary embodiment of the present disclosure; and

FIG. 3 is a flowchart illustrating a diagnostic process according to an exemplary embodiment of the present disclosure in more detail.

The drawings described herein are for illustration purposes only and are not intended to limit the scope of the present disclosure in any way.

### DETAILED DESCRIPTION

The terms and words used in the following description and appended claims are not necessarily to be construed in an ordinary sense or a dictionary meaning, and may be appropriately defined herein to be used as terms for describing the present disclosure in the best way possible. Such terms and words should be construed as meaning and concept consistent with the technical idea of the present disclosure. The embodiments described in this specification and the configurations shown in the drawings are merely preferred embodiments of the present disclosure and are not intended to limit the technical idea of the present disclosure. Therefore, it should be understood that there may exist various equivalents and modifications which may substitute the exemplary embodiments at the time of filing of the present application.

For a more clear understanding of the features and advantages of the present disclosure, the present disclosure will be described in detail with reference to the accompanied drawings. It is to be noted that the same components are designated by the same reference numerals throughout the drawings. In the following description and the accompanied drawings, detailed descriptions of well-known functions or configuration that may obscure the subject matter of the present disclosure will be omitted for simplicity. Also, some components in the accompanying drawings may be exaggerated, omitted, or schematically illustrated for the same reason, and the size of each component does not fully reflect its actual size.

The present disclosure proposes a method and apparatus for assisting the health of a user using the history of health condition of the user. The health care according to the present disclosure includes a diagnosis of a skin problem, a diagnosis of an abnormality in the endocrine system showing a symptom through a skin, an exoskeleton measurement and diagnosis, a posture measurement, and the like. The health care apparatus according to an exemplary embodiment of the present disclosure may be any device equipped with a camera installed at home or in an office or a portable device such as a smartphone. Such a device may be used in conjunction with a mirror which is frequently used in everyday life to enable a periodic shooting and a shooting at a constant or variable shooting distance and angle.

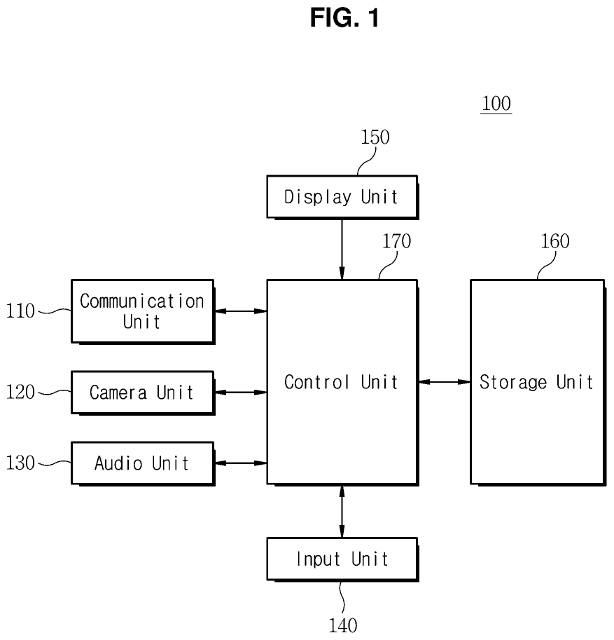

A configuration of a health care apparatus for assisting the health care of a user according to an exemplary embodiment of the present disclosure will now be described with reference to FIG. 1. FIG. 1 is a block diagram of a health care apparatus according to an exemplary embodiment of the present disclosure.

Referring to FIG. 1, a health care apparatus 100 according to an exemplary embodiment of the present disclosure includes a communication unit 110, a camera unit 120, an audio unit 130, an input unit 140, a display unit 150, and a storage unit 160, and a control unit 170.

The communication unit 110 communicates with other devices through a network to acquire external information. Examples of the external information may include weather information, statistics information for other users, rule-based guide literature data, and the like. For example, the communication unit 110 may collect the weather information by accessing a server providing weather information under the control of the control unit 170. The communication unit 110 may include a radio frequency (RF) transmitter suitable for up-converting a frequency band of a transmitted signal and amplifying the transmitted signal and a RF receiver suitable for low-noise-amplifying a frequency band of a received signal and down-converting the received signal. The communication unit 110 may include a modem suitable for modulating the transmitted signal and demodulating the received signal.

The camera unit 120, which captures an image. includes an image sensor. The image sensor receives light reflected by an object and converts the light into an electrical signal. The image sensor may be implemented based on a charge coupled device (CCD) or a complementary metal-oxide-semiconductor (CMOS) image sensor. The camera unit 120 may further include an analog-to-digital converter, and may convert an analog signal output by the image sensor into a digital signal to output the digital signal to the controller 170.

The audio unit 130 includes an audio input module (not shown) receiving an audio signal such as a voice to provide a received voice signal to the controller 170 and an audio output module (not shown) outputting an output audio signal provided by the controller 170. The audio input module may be at least one microphone. According to the exemplary embodiment, the microphone may receive a voice of the user including the user's answer during a conversation and to provide to the controller 170. The audio output module may include a speaker. According to the exemplary embodiment, the audio output module may output a query message to the user as a voice under the control of the controller 170.

The input unit 140 receives a key input of the user suitable for controlling the health care apparatus 100, generates an input signal corresponding to the key input, and provides the input signal to the controller 170. The input unit 140 may include multiple keys for manipulating the health care apparatus 100. In case that the input unit 140 is implemented based on a touch panel, the multiple keys may be embodied by keys displayed on the touch panel. Moreover, in a case where all the functions of the input unit 140 is performed by the touch panel only, the input unit 140 may be integrated with the display unit 150 into a touch panel screen.

The display unit 150 visually provides the user with a menu of the health care apparatus 100, input data, function settings information, and various other information. The display unit 150 may display various screens such as a boot screen, a standby screen, and a menu screen of the health care apparatus 100. In particular, the display unit 150 performs a function of outputting a variety of information, on the screen, according to an embodiment of the present disclosure. The display unit 150 may be implemented by a liquid crystal display (LCD), an organic light emitting diode (OLED), an active matrix organic light emitting diode (AMOLED), or the like. Meanwhile, the display unit 150 may be implemented by a touch panel screen as mentioned above. In such a case, the touch panel screen includes a touch sensor. The touch sensors detect a touch input of the user. The touch sensor may be implemented by a touch sensing sensor such as a capacitive overlay type sensor, a pressure type sensor, a resistive overlay type sensor, or an infrared beam type sensor, or may be implemented by a pressure sensor. Another kinds of sensors capable of sensing a contact or pressure applied by the user can be used as the touch sensor of the present disclosure. When the touch sensor senses a touch input of the user, the touch sensor generates a touch sensing signal to provide to the controller 170.

The storage unit 160 stores a program and data required for the operation of the health care apparatus 100. In particular, the storage unit 160 may accumulatively store user data (i.e. history information) generated as the use of the health care apparatus 100 progresses. In addition, the storage unit 160 may store personal basic health status characteristics, biorhythms, preference information (e.g. food, clothing, etc.) of the user. Also, the storage unit 160 may additionally store external information received through the communication unit 110. As mentioned above, the external information may include the weather information, the statistics information for other users, the rule-based guide literature data, weather information, other user statistics information, rule-based guide literature data, and the like. The data stored in the storage unit 160 may be deleted, changed, or added according to the user's manipulation of the health care apparatus 100.

The controller 170 controls overall operation of the health care apparatus 100 and signal flows among the internal blocks of the health care apparatus 100, and performs data processing operations. The controller 170 controls various functional operations of the health care apparatus 100. The controller 170 may include a central processing unit (CPU), a digital signal processor (DSP), or the like. The operation of the control unit 170 will be described in more detail below.

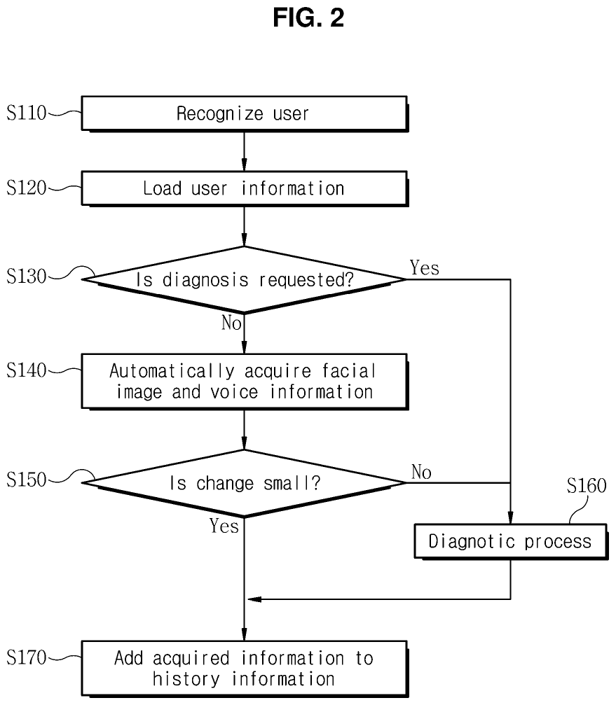

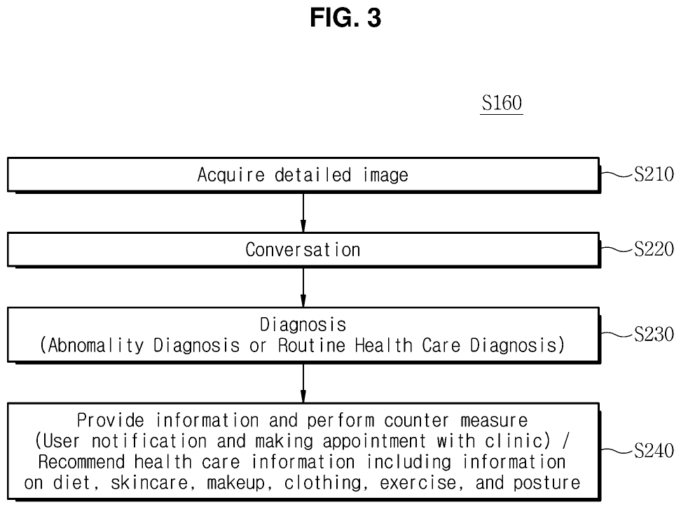

Next, a user health management method according to an exemplary embodiment of the present disclosure will be described with reference to FIGS. 2 and 3. FIG. 2 is a flowchart illustrating the user health management method according to an exemplary embodiment of the present disclosure, and FIG. 3 is a flowchart illustrating a diagnostic process according to an exemplary embodiment of the present disclosure in more detail.

Referring to FIG. 2, the control unit 170 recognizes the user in operation S110. According to an exemplary embodiment of the present disclosure, when the user inputs an ID, a password, or the like into the input unit 140, the control unit 170 may recognize that the user has logged in by comparing the ID and password entered by the user with a registered ID and password previously stored in the storage unit 160. According to another exemplary embodiment, the control unit 170 may photograph the user through the camera unit 120 to recognize the user through a facial recognition, i.e. by comparing facial features selected from an image acquired by the camera unit 120 with facial features stored in the storage unit 160. According to yet another exemplary embodiment, the control unit 170 may collect the user's voice through the audio unit 130 to recognize the user through a voice recognition, i.e. by comparing the voice acquired through the audio unit 130 with the voices stored in the storage unit 160.

In operation S120, the control unit 170 loads information about the user recognized in the operation S110 from the storage unit 160. The user information includes the history information of the user.

In operation S130, the control unit 170 determines whether the user requests a diagnosis through the input unit 140. In case that the user requests the diagnosis, the control unit 170 performs an operation S160 to perform a diagnostic process. On the other hand, in case that the user does not request the diagnosis, the procedure proceeds to an operation S140.

In the operation S140, the control unit 170 automatically photographs the user's face and collects the user's voice through the camera unit 120 and the audio unit 130, respectively, and performs an analysis of the facial and voice information. The operation S140 may be performed automatically at regular time intervals every day or when the user comes within a shooting range. The analysis is performed by comparing facial image and voice tone of the user which are collected newly every day with corresponding information existing in the history information to detect a change in the facial image or the voice tone.

In step S150, the control unit 170 determines whether the change in the facial image or the voice tone is small compared to the previous information existing in the history information. In case that the change is determined to be small compared to the existing history information, the control unit 170 performs an operation S170 to add the facial and voice information of the user acquired in the operation S140 to the history information. On the other hand, in case that the change is larger than a predetermined level, the procedure proceeds to the operation S160 to perform the diagnostic process.

As described above, the control unit 170 performs the diagnostic process in the operation S160 when the user requests the diagnosis in the operation S130 or when the control unit 170 determines that the change in the facial image or the voice tone is larger than the predetermined level in the operation S150 during an automatic analysis. The diagnostic process of the operation S160 will now be described in more detail with reference to FIG. 3.

Referring to FIG. 3, the controller 130 captures a detailed image centering on a body part to be diagnosed (e.g. the user's face) through the camera unit 120 in operation S210.

In operation S220, the control unit 170 loads a query stored in the storage unit 160 to ask at least one question to the user through the audio unit 130 and collect a voice response of the user to the question through the audio unit 130.

In operation S230, the control unit 170 analyzes and diagnoses the state of the user by comparing the detailed image acquired in the operation S210 and the response of the user to the question and the voice information acquired in the operation S220 with corresponding information in the history information stored in the storage unit 160. The diagnosis includes an abnormality diagnosis and a routine health case diagnosis.

During a process of the abnormality diagnosis, the control unit 170 may load, from the storage unit 160, (1) underlying medical conditions of the user including any drug-in-use and underlying disease information, and (2) the history information. Then, the control unit 170 determines whether there is any abnormality by comparing the detailed image information, the voice information extracted from the response of the user during a conversation between the user and the health care apparatus, and the response with the previous image existing in the history information, the previous voice existing in the history information, and the underlying medical condition information, respectively. At this time, the control unit 170 can diagnose dermatological diseases in addition to a simple skin problem. Also, the control unit 170 may perform a diagnosis of an abnormality in the endocrine system that shows a symptom through a skin, an exoskeleton measurement and diagnosis, a posture measurement, and the like.

During a process of the routine health case diagnosis, the control unit 170 may load, from the storage unit 160, (1) underlying medical conditions of the user, (2) the history information. and (3) personal information of the user and external information. Here, the personal information may include biorhythm of the user, user preference regarding food and clothes, history information on previous recommendations, and the like. The external information may include weather information, the statistics information for other users, the rule-based guide literature data, and the like. Then, the control unit 170 determines a current state of the user by comparing the detailed image information and the voice information extracted from the response of the user during the conversation between the user and the health care apparatus with the previous image and the previous voice existing in the history information, respectively, and comparing the response of the user with the underlying medical condition information, the personal information, and the external information. At this time, the control unit 170 may check today's diet, skin care, makeup, clothing, exercise, and posture of the user.

In operation S240, the control unit 170 provides necessary information to the user or perform a counter measure required for the user according to the diagnosis result.

According to an embodiment, when no abnormality is found in the abnormal diagnosis process, the control unit 170 estimates a period required for regeneration to a normal condition and informs the estimated regeneration period to the user through the display unit 150. For example, in the case of a skin overexposed to the sun, the control unit 170 may estimate a regeneration period based on skin recovery phase and current the skin condition information of the user previously stored in the history information to inform the estimated regeneration period to the user. Meanwhile, in case that an abnormality is detected during the abnormality diagnosis process, the control unit 170 may automatically perform a procedure of making an appointment with a clinic through the communication unit 110.

According to another embodiment, the control unit 170, which have found the current condition of the user during the routine health care diagnosis process, may recommend appropriate health care information for the user under the current state by using the personal information of the user and the external information. Recommended health care information may include information on the diet, skincare, makeup, clothing, exercise, and posture.

Referring back to FIG. 2, after the diagnosis process is completed, the control unit 170 proceeds to operation S170 to update the history information in the storage unit 160 by adding new history data. The history information includes diagnostic information, hospital linkage information, and health care recommendation details in addition to the facial and voice information of the user.

As described above, the present disclosure can improve an accuracy of a diagnosis by analyzing the image and voice of the user and the conversation with the user and combining the analysis results. Since symptoms appearing on a human body alone are often insufficient for accurately identifying the condition of the human body, the present disclosure improves the accuracy of the diagnosis by reflecting the analysis of the conversation with the user similarly to a medical examination by interview in a hospital. For example, if edema was found in the image analysis and it was found in the conversational analysis that the user ingested excess salt through food, the apparatus of the present disclosure can diagnose the problem of the user as transient edema as a result of a combined diagnosis and recommend dietary control to the user.

In addition, the present disclosure enables to diagnose a body rhythm and health condition of the user by detecting the tone of the voice and the change thereof which may be acquired from voice information.

The present disclosure enables to preemptively identify and cope with abnormal symptoms by automatically analyzing the routine or everyday life of the user and building up a history of such information.

Through the diagnosis using the recently acquired information and the history information, the present disclosure can perform a customized diagnosis of the abnormal conditions for the user, enable the user to perform health care related activities routinely, and provide the recommendations for enhancing health conditions of the user.

The method according to exemplary embodiments of the present disclosure may be implemented in a form of a program readable by various computing devices and may be recorded in a computer-readable storage medium. Here, the storage medium may store program instructions, data files, data structures, and a combination thereof. The program instructions recorded on the storage medium may be those specifically designed and constructed for the present disclosure or may be those available to those skilled in the art of computer software. Examples of the storage medium may include magnetic media such as a hard disk, a floppy disk, and a magnetic tape, optical media such as a compact disk read only memory (CD-ROM) and a digital video disk (DVD), magneto-optical medium such as a floptical disk, and semiconductor memories such as ROM, RAM, and the flash memory, erasable and programmable ROM (EPROM), and electrically erasable and programmable ROM (EEPROM), all of which are hardware devices suitable for storing the computer program instructions and data. The program instructions may be coded in, for example, a machine language or a high-level language that may be complied or interpreted by a compiler or an interpreter, respectively. The hardware device may be configured to operate similarly to one or more software modules to perform the operations required for the present disclosure, and vice versa.

The description of the disclosure is merely exemplary in nature and, thus, variations that do not depart from the substance of the disclosure are intended to be within the scope of the disclosure. Such variations are not to be regarded as a departure from the spirit and scope of the disclosure. Thus, it will be understood by those of ordinary skill in the art that various changes in form and details may be made therein without departing from the spirit and scope as defined by the following claims.

What is claimed is:

1. A method of managing health of a user in a health care apparatus, comprising:
recognizing the user and determining whether to start a diagnosis process for the user according to a prescribed condition by the health care apparatus;
loading history information for the user from a storage unit of the health care apparatus after the diagnosis process begins;
acquiring an image and a voice of the user by the health care apparatus;
performing the diagnosis process, by the health care apparatus, by analyzing the history information and the image and voice information of the user, and providing reference information to the user or performing a corresponding action depending on a diagnosis result; and
updating the history information stored in the storage unit by adding the image and voice information of the user, the diagnosis result, and the reference information or information of the corresponding action.

2. The method of claim 1, wherein the determining whether to start the diagnosis process for the user comprises:
photographing the image of the user and collecting the voice of the user, by the health care apparatus, to analyze a change in the image or the voice with reference to a previous image or a previous voice, respectively, existing in the history information;
determining, by the health care apparatus, whether the change in the image or the voice is small compared to the previous image or the previous voice; and
performing the diagnosis process, by the health care apparatus, when the change is determined to be larger than a predetermined threshold and updating the history information by adding the image and voice information of the user, by the health care apparatus, when the change is determined to be smaller than the predetermined threshold.

3. The method of claim 1, wherein the providing the reference information to the user or performing the corresponding action depending on the diagnosis result comprises:
acquiring the image and voice of the user, and conversational information for a conversation between the user and the health care apparatus by the health care apparatus;
loading underlying medical condition information of the user and the history information for the user from the storage unit of the health care apparatus;
determining whether there is any abnormality, by the health care apparatus, by comparing the image, the voice, and the conversational information with a previous image existing in the history information, a previous existing in the history information, and the underlying medical condition information, respectively; and
estimating, by the health care apparatus, a period required for regeneration to a normal condition and informing the estimated regeneration period to the user when no abnormality is found in the diagnosis process and making an appointment, by the health care apparatus, with a clinic when an abnormality is detected during the diagnosis process.

4. The method of claim 1, wherein the providing the reference information to the user or performing the corresponding action depending on the diagnosis result comprises:
acquiring the image and the voice of the user, and conversational information for a conversation between the user and the health care apparatus by the health care apparatus;
loading underlying medical condition information of the user, the history information for the user, personal information of the user, and external information from the storage unit of the health care apparatus;
determining a current condition of the user, by the health care apparatus, by comparing the image and the voice of the user with a previous image and a previous voice existing in the history information, respectively, and comparing the conversational information with the underlying medical condition information, the personal information, and the external information; and
recommending, by the health care apparatus, one or more of health care information selected from a group of information on diet, skincare, makeup, clothing, exercise, and posture suitable for the user and determined based on the personal information and the external information according to a current condition of the user.

5. A health care apparatus comprising:
a memory for storing history information for a user and instructions;
an audio unit acquiring a voice of the user;
a camera capturing an image of the user; and
a processor executing the instructions stored in the memory, the instructions when executed by the processor causing the processor to:
determine whether to start a diagnosis process for the user according to a prescribed condition when the user is recognized;
load the history information for the user from the memory after the diagnosis process begins;
acquire the image and the voice of the user through the audio unit and the audio unit, respectively;
perform the diagnosis process by analyzing the history information and the image and voice information of the user, and provide reference information to the user or perform a corresponding action depending on a diagnosis result; and
update the history information stored in the memory by adding the image and voice information of the user, the diagnosis result, and the reference information or information of the corresponding action.

6. The health care apparatus of claim 5, wherein the instructions when executed by the processor causes the processor to:
acquire the image of the user through the camera and acquire the voice of the user and conversational information for a conversation between the user and the health care apparatus through the audio unit to analyze a change in the image or the voice with reference to a previous image or a previous voice, respectively, existing in the history information;
determine whether the change in the image or the voice is small compared to the previous image or the previous voice; and
perform the diagnosis process when the change is determined to be larger than a predetermined threshold and update the history information by adding the image and voice information of the user when the change is determined to be smaller than the predetermined threshold.

7. The health care apparatus of claim 5, wherein the instructions when executed by the processor causes the processor to:
acquire the image of the user through the camera and acquire the voice of the user and conversational information for a conversation between the user and the health care apparatus through the audio unit;
load underlying medical condition information of the user and the history information for the user from the memory;
determine whether there is any abnormality by comparing the image, the voice, and the conversational information with a previous image existing in the history information, a previous existing in the history information, and the underlying medical condition information, respectively; and
estimate a period required for regeneration to a normal condition and informing the estimated regeneration period to the user when no abnormality is found in the diagnosis process and make an appointment with a clinic when an abnormality is detected during the diagnosis process.

8. The health care apparatus of claim 5, wherein the instructions when executed by the processor causes the processor to:
acquire the image of the user through the camera, the voice, and conversational information for a conversation between the user and the health care apparatus through the audio unit;
load underlying medical condition information of the user, the history information for the user, personal information of the user, and external information from the memory;
determine a current condition of the user by comparing the image and the voice of the user with a previous image a previous voice existing in the history information, respectively, and comparing the conversational information with the underlying medical condition information, the personal information, and the external information; and
recommend one or more of health care information selected from a group of information on diet, skincare, makeup, clothing, exercise, and posture suitable for the user and determined based on the personal information and the external information according to a current condition of the user.
